(19) United States
(12) Patent Application Publication
(10) Pub. No.: US 2012/0202972 A1
(43) Pub. Date: Aug. 9, 2012

(54) Motor proteins propelling nano-scale devices and systems

(75) Inventors: Parviz Famouri, Morgantown, WV (US); Yongkuk Lee, Morgantown, WV (US); Lenin Leon, Morgantown, WV (US)

(73) Assignee: West Virginia University

(21) Appl. No.: 13/506,059

(22) Filed: Mar. 23, 2012

Related U.S. Application Data
(62) Division of application No. 12/592,920, filed on Dec. 4, 2009.
(60) Provisional application No. 61/201,077, filed on Dec. 5, 2008.

(51) Int. Cl. C07K 17/00; C07K 1/04

(52) U.S. Cl. 530/367; 530/408

(57) ABSTRACT

An embodiment can be the use of motor proteins for cargo loading and transport in nano-devices and systems. One embodiment of the use of motor proteins can be adding biotin-binding proteins to a substrate by patterning, binding biotinylated F-actin to the biotin-binding proteins, aligning the bound F-actin in a preferred direction using a flow field, and using myosin coated particles to transport items attached to the particle throughout the substrate. Another embodiment of the use of motor proteins can be adding biotin-binding proteins to a substrate by patterning, adding a flow field, injecting F-actin so that the F-actin is bound and aligned simultaneously, and using myosin coated particles to transport items attached to the particle throughout the substrate. In either embodiment the F-actin can be capped with a biotinylated cap before binding to the biotin-binding proteins.

### CROSS-REFERENCE TO RELATED APPLICATIONS

This application is a Divisional from parent application No. 12/592,920 filed on Dec. 4, 2009 which claims priority from provisional patent application No. 61/201,077 filed on Dec. 5, 2008.

### STATEMENT REGARDING FEDERALLY SPONSORED RESEARCH OR DEVELOPMENT

This invention was made with government support under Grant No. ECS0403742 awarded by the National Science Foundation. The United States government has certain rights in the invention.

### REFERENCE TO SEQUENCE LISTING, A TABLE, OR A COMPUTER PROGRAM LISTING COMPACT DISC APPENDIX

### Brief Description of the Several Views of the Drawing

FIG. 1 is a schematic representation of conjugation.

FIG. 2 is a schematic representation of a process of biomolecular patterning.

FIG. 3 is schematic representation of F-actin with biotinylated actin capping proteins.

FIG. 4 is a schematic representation of the electrode structure for ACEO pumps of straight flows (a) top view and (b) side view and a schematics of electrode arrays to generate arc shaped flow field.

FIG. 5 is (a) a schematic representation of flow cell and (b) a schematic representation of depicting the selective assembly of F-actin by biotinylated capping protein with specific structural polarity on desired locations.

FIG. 6 is a schematic representation of successful movement of myosin coated beads along F-actin pathways.

FIG. 7 is a schematic representation of patterned F-actin with structural polarity.

FIG. 8 is a schematic representation of a bio-actuator with rotational movement.

FIG. 9 is a schematic representation of the production of localized fields.

### DETAILED DESCRIPTION OF THE INVENTION

An embodiment can be the use of motor proteins for cargo loading and transport in nano-devices and systems. One embodiment of the use of motor proteins can be adding biotin-binding proteins to a substrate by one or more patternings, binding biotinylated F-actin to the biotin-binding proteins, aligning the bound F-actin in a preferred direction using one or more flow fields, and using one or more myosin coated particles to transport items attached to the particle throughout the substrate with a chemical fuel. Another embodiment of the use of motor proteins can be adding biotin-binding proteins to a substrate by one ore more patternings, adding one or more flow fields, injecting F-actin so that the F-actin is bound and aligned simultaneously, and using one or more myosin coated particles to transport items attached to the particle throughout the substrate. In either embodiment the F-actin can be capped with a biotinylated cap before binding to the biotin-binding proteins.

In this application biotin-binding proteins can be any protein capable of binding biotin such as streptavidin, avidin, neutravidin, and any other known to one skilled in the art. The substrate can be any suitable substrate such as glass, quartz, a plastic, or any substrate known to one skilled in the art. The biotin-binding protein or biotin can be bound to the substrate by conjugation techniques. Conjugation techniques can be accomplished by physical or chemical modifications on biotin-binding protein, biotin or substrate. Modifications can be creating specific functional groups such as amine, aldehyde, carboxylate, hydroxyl and any other known to one skilled in the art on biotin-binding protein, biotin or substrate. Those functional groups can be utilized to form non-covalent or covalent binding between biotin-binding protein and substrate or biotin and substrate. Patterning techniques which help biotin-binding protein or biotin bound on selected area on the substrate can be accomplished by either the use of UV sensitive polymers that change the surface properties of a substrate as forming functional groups on the surface in exposure of UV, the use of a photo-biotin exposed to UV to bind the biotin covalently at the sites containing C-H or N-H bonds, scanning probe lithography techniques such as Dip-Pen Nanolithography or soft and conventional lithography techniques on the surfaces with specific functionalities. In any patterning method the bound biotin-binding protein or the bound biotin can be confirmed by the use of conventional conformation techniques such as fluorescent dye or quantum dot labeling.

### Example 1

### Conjugation

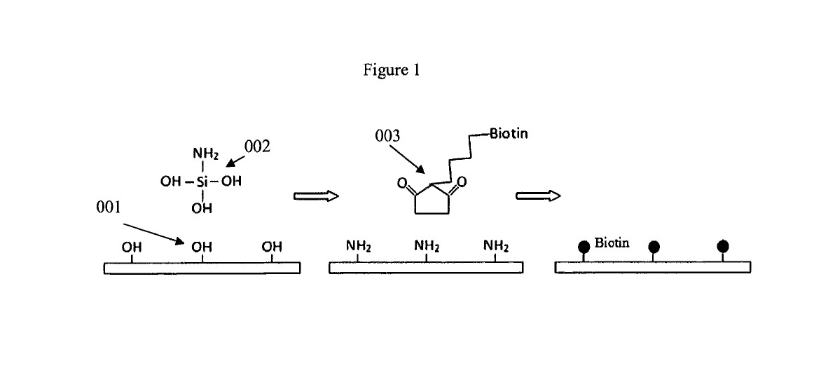

The conjugation of these can be performed by using Aminopropyltriethoxysilane (APTES) and N-Hydroxysuccinimide (NHS) conjugated biotin as shown in FIG. 1 where 001 are Hydroxyl groups, 002 is APTES, and 003 is NHS-biotin. Aminopropyltriethoxysilane (APTES) solution (5% APTES, 5% deionized water and 90% ethanol) is prepared. The APTES solution is injected onto the clean glass slide where most organic matter is removed and hydroxyl groups are formed to form amine groups on the surface of the clean glass slide for 1 hour. The glass slide is washed with ethanol and 2 mg/ml of NHS conjugated biotin in anhydrous dimethylformamide (DMF) is followed. The glass slide is washed with B-PBS buffer (150 mM NaCl and 100 mM Na₂HPO₄ at pH 7.2). 100 ug/ml of fluorescent labeled streptavidin is incubated for 1 hour and it is observed to confirm biotin conjugation on the glass slide.

### Example 2

### Patterning

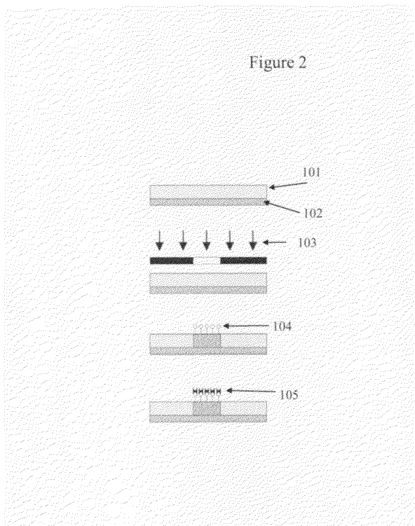

The patterning of these can be performed by using soft lithography as shown in FIG. 2. Micropattern is developed on 3″ silicon wafer using SU-8 25. After polydimethylsiloxane (PDMS) is poured on the pattern and cured in an oven at 100° C. for 4 hours. The cured PDMS is soaked with ATPES solution (5% APTES, 5% deionized water and 90% ethanol) 101 and then the micropatten is stamped on the clean glass substrate forming hydroxyl groups 102 forming the APTES layer patterned substrate 103. NHS conjugated biotin can be immobilized on the stamped areas 104 as the solution containing 2 mg/ml of NHS conjugated biotin in anhydrous DMF is incubated on the patterned substrates at room temperature for 2 hours. Then, 100 ug/mL streptavidin will be incubated on the biotin patterned substrate so that F-actin biotinylated at its structural minus end can be bound 105. To confirm immobilization of biotin, fluorescent dye labeled streptavidin can be used. Microdevices can be incubated in myosin solution. This procedure allows microdevices to be coated with myosin before being built with the F-actin patterned substrate. UV exposure can be used as a switch for bio-actuator motion. Caged ATP is employed as chemical fuel for bio-actuator which is biologically active upon UV light exposure. After creating F-actin patterns, the actin crosslinking proteins such as fascin, fimbrin, a-actinin and any other protein that can crosslink actins can be used to keep F-actin patterns stable on the surface of the biomolecular patterned substrate from any disturbance, if necessary.

No matter the embodiment of patterning used biomolecules with different geometries can be patterned. Straight lines can be developed to create a pathway where a layer of actin can be placed along with myosin coated particles carrying a specific cargo protein. These particles can then be propelled by the interaction of myosin-actin biomolecular motors. To ensure an optimal transportation track for cargo protein, it is necessary to ensure the uniformity of the molecular patterning (which depend on the specific binding of the species in the selected hydrophilic modified areas), and to optimize the dimensions of the track based upon the kind of microparticles being used in the transportation of the cargo protein. For curvatures it will be necessary to minimize actin filaments being outside the limits of the actin pathway structure. It will be necessary to control the length of F-actin. Capping proteins can regulate the dynamics of actin filaments. The use of capping proteins in areas of high curvature can minimize the issue of actin filaments outside the pathway.

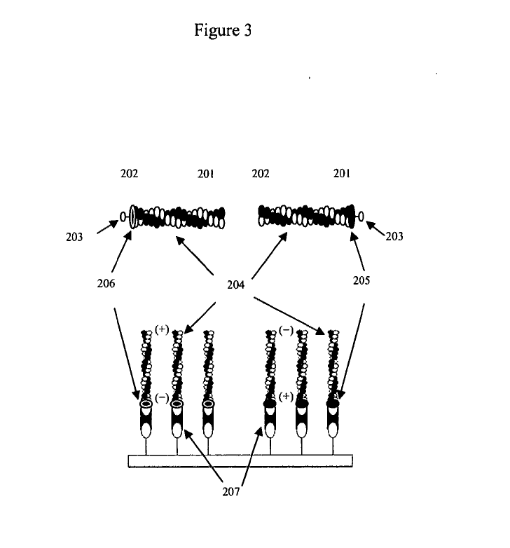

Capping proteins can act as a tool to regulate the dynamics of F-actin as shown in FIG. 3. In-vitro biotinylation linkages can be used for substrate attachment of actin filaments when the cap is biotinylated and added to the actin. Actin has a positive 201 and negative end 202 and capping the actin with a biotinylated protein 203 and binding to the biotin-binding protein can regulate the structural polarity of the actin 204. Actin capping proteins such as CapZ, severin, and any other protein that can cap the plus end can be used to cap the plus end of the actin 205. The minus end can be capped by proteins such as gelsolin, villin, and any protein that can be used to bind the minus end of actin 206. The capped actin can then be bound to the biotin-binding protein 207. Gelsolin can be used to control the length of actin, due to its efficiency of severing the filaments by changing the levels of Ca²⁺ in the solution. The use of both positive and negative capping proteins allows for two way tracks with one directional flow field.

### EXAMPLE

### Biotinylation of Actin Capping Proteins

The preparation of biotinylated actin capping proteins can be accomplished by any functional group conjugated biotin which can be bound covalently or non-covalently on actin capping proteins. For example, actin capping protein is dialyzed against a crosslinking phosphate buffer (B-PBS buffer, 150 mM NaCl and 100 mM Na₂HPO₄ at pH 7.2). NHS-PEG₄-Biotin is dissolved in deionized water immediately before use at a concentration of 2 mg/ml. The dissolved biotin solution and dialyzed protein solution are mixed with a concentration ratio of 60:1 for 2 hours in ice. During incubation, biotin is bound covalently on amine group in actin capping protein due to covalent reaction between NHS group and amine group. Unattached biotins are removed by dialysis in actin capping protein storage buffer and biotinylated actin capping protein is stored in −80° C. until use.

### Example 2

### F-Actin Capping and Flow Fields

The preparation of the sample of F-actin with biotinylated actin capping proteins can be accomplished by incubating F-actin with biotinylated actin capping protein for 1 hour in ice with a concentration ratio of 1:2 to get the complex of biotin-capping protein-actin ready for assembly.

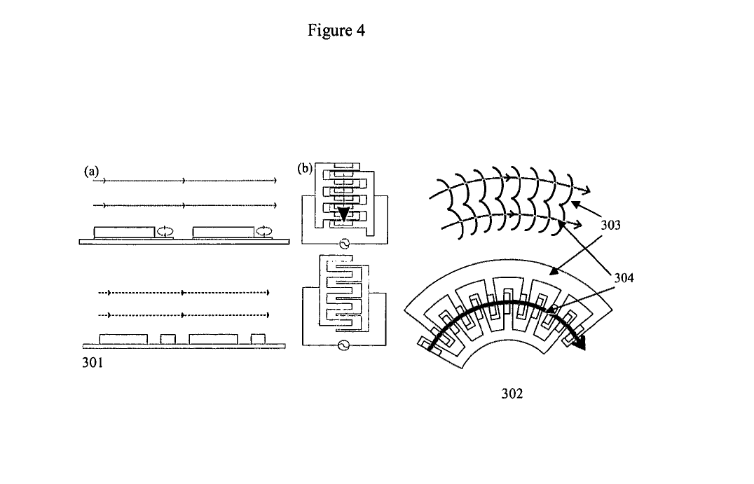

Flow field will be utilized to lay and align assembled F-actin along the desired direction. Flow field devices can be any standard device capable of controlling velocity and the density of a fluid as functions of position and time such as AC electro-osmosis (ACEO) pumps employed to generate localized flow fields. The field flow device can generate straight and arc shaped flow field by using an AC electric field FIG. 4. A field flow device can generate straight streamline flow fields 301. The size of electrodes will be varied according to the width of actin tracks. Field flow devices can also be used to generate arc shaped flow fields 302 by utilizing electrode arrays in a curved manner 303 to control the flow field 304. The patterning layer of actin and the substrate patterned electrodes are separate structures that are aligned before the generation of a localized field either applied in turn or simultaneously. One important factor to consider is the separation of these two structures. Polydimethylsiloxane (PDMS) can be used to accomplish the required separation between them. The alignment of the biopatterned actin and the electrodes can be accomplished by a series of alignment marks in both substrates, during the photolithography process that both structures undergo, in order to have an easy way to aligned them during the integration of the system. The result can yield the unidirectional movement of myosin coated particles as the actin is fully aligned in the same direction.

### Example 3

### Alignment of F-Actin

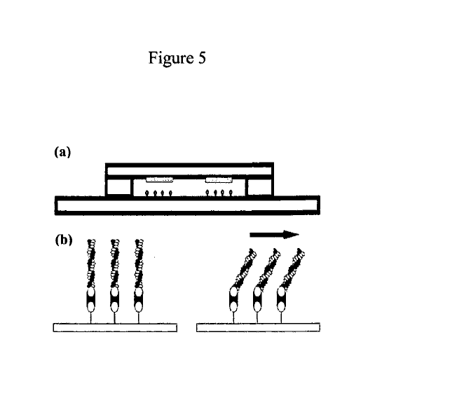

The flow cell can be constructed on the substrate where electrode arrays are fabricated and the substrate where F-actin is patterned FIG. 5. PDMS can be placed between the two substrates for an effective flow field. Before patterning F-actin, streptavidin patterning can be performed. Then the flow cell can be washed out by M-buffer solution containing 25 mM KCl, 2 mM MgCl₂, 0.2 mM CaCl₂ and 25 mM Imidazole at pH 7.0. The capped F-actin by biotinylated capping protein can be then injected into flow cell and the electrodes can be energized to align the actin. Alternatively the electrode arrays can energized, the capped F-actin can be injected into flow cell until the actin track is fully assembled. To confirm F-actin is patterned correctly, fluorescent labeled F-actin can be used.

A myosin coated particle can be any transport devices such as a bead, nanowire, or nanotube. Any particle with a hydrophobic surface can be used for the particle for myosin coat attachment. Some commercial spherical beads with different diameters are available such as tosyl-activated polystyrene beads. Moreover, microfabrication can also create various shaped particles of SiO₂ with various sizes. Cr or Au thin layer can be deposited on a substrate. A SiO₂ layer with desired thickness can be deposited on the metal thin layer by using PECVD. The metal layer can be etched after photolithography and SiO₂ etching process are performed to fabricate desired shaped SiO₂ particles. By using filtration and centrifugation process, SiO₂ particles can be gathered and coated by hydrophobic thin film. The choice of a specific type of particle will depend on the application. The velocity of the particle is dependent on the shape of the particle and also dependent on the material, weight, and area size where myosin interacts with the F-actin rail. The use of multiple particles of differing characteristics such as motility speed, electrical properties, size, and capacitance change can be utilized to identify and sort distinct molecules in an assay. The motion due to interaction of myosin and actin can be observed by coating the particles with identification mechanisms such as fluorescent dyes. In order to increase the specificity of protein cargo collection, beads can be decorated with antibodies to those proteins. Antibodies can be covalently coupled using bifunctional coupling reagents that react with carboxyl or amine groups on bead surface. Fluorescent polystyrene beads with a narrow size distribution and functionalized surface, including amine and carboxylic acid functional groups can be further utilized. Commercially derivatized antibodies can be obtained to bind covalently to the bead. Conventional methods such as incubation can be used for antibody or protein binding on functionalized beads. Detachment of proteins from the beads will be dependent on the proteins/antibodies being transported. Control of pH or salt concentration in solution is a kind of example to detach proteins from the beads.

### Example 4

### Myosin Coated Particles

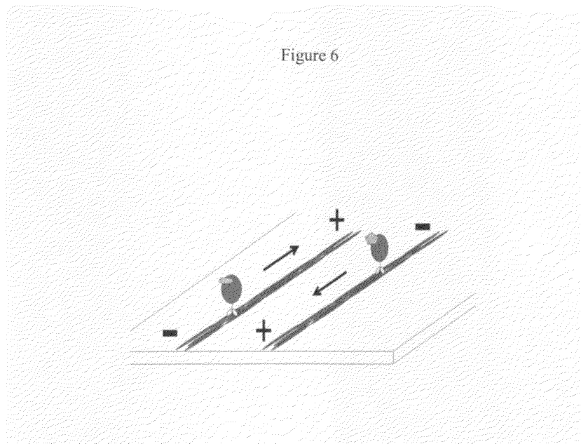

After preparation of hydrophobic particles, the particles can be incubated with enough of concentrated stock solution of myosin such as 1.0 mg/ml in M-buffer to give the desired final concentration of myosin on particles. Particles can be incubated in myosin solution on ice at least 1 hour before they are used in motility assay. The movement of myosin coated particles walking along F-actin can be observed and the velocity measured FIG. 6.

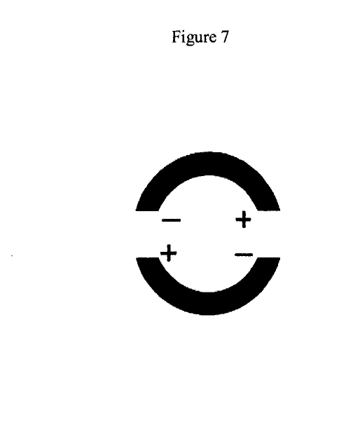

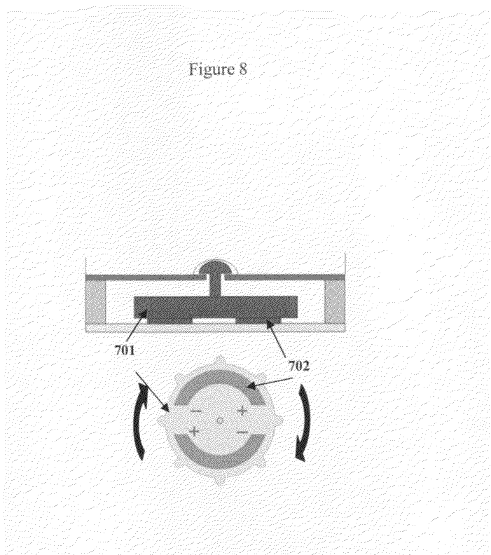

The generation of rotational movement can be performed with specific shaped F-actin pattern such as the use of two circular arc-shaped F-actin tracks with structural polarity will be patterned in this work FIG. 7. The circular arc-shaped F-actin pattern is a kind of the geometry pattern which can provide continuous clockwise or counter-clockwise rotational movement of myosin coated microdevices such as toothed wheel shaped micro-gears in existence of ATP FIG. 8. A myosin coated micro-gear 701 and an arc-shaped F-actin track 702 can be used to create a bioactuator with rotational movement. The approach of a myosin coated toothed-wheel can be used as a micro-engine in bio-MEMs (Micro Electromechanical System) applications. The result can be a patterned actin rail system for a micro-engine.

To minimize the possibility of undesired binding of F-actin on streptavidin coated surfaces during the process, F-actin patterning process can be performed separately for adjacent patterns. When creating two adjacent arc-shaped F-actin patterns two localized flow fields will be necessary.

### Example 5

### Creation of Localized Fields That Do Not Disturb One Another

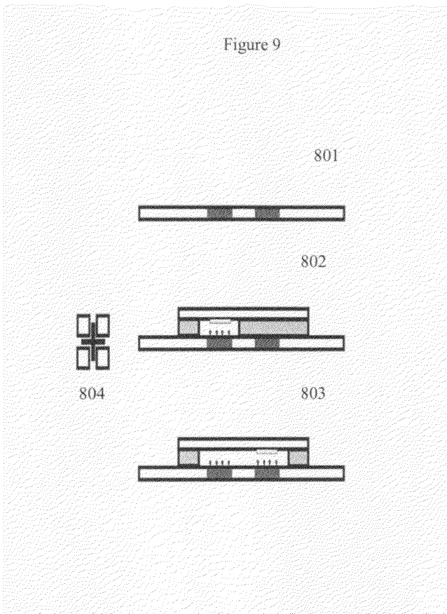

UV sensitive polymer can be coated on a glass substrate by spin coating as in FIG. 9. Then, UV light can be exposed on areas which will be patterned 801. The UV light can force exposed areas to be hydrophilic using the methods previously disclosed. A plurality of electrode array substrates able to generate localized flow field can be utilized. A PDMS channel can be molded on one electrode substrate 802 and the electrode substrate can then be placed on the glass substrate to form a microchannel. The microchannel allows working in just one biomolecular patterned area while other adjacent patterning areas can be covered by PDMS keeping any protein from binding on the covered area. F-actin then will be patterned with localized flow field on the uncovered hydrophilic area. After patterning F-actin with the desired arrangement, the flow cell can be completely washed away. Then another electrode substrate can be placed on the non-protein coated hydrophobic area 803 and a repetition of the F-actin patterning process can be completed for a second pattern. The substrate of electrode arrays for generating flow fields can be taken off and microdevices can be built on F-actin patterned area with precise alignment after patterning F-actin with the desired arrangement 804. This process can be useful to create any adjacent F-actin patterns.

These terms and specifications, including the examples, serve to describe the invention by example and not to limit the invention. It is expected that others will perceive differences, which, while differing from the forgoing, do not depart from the scope of the invention herein described and claimed. In particular, any of the function elements described herein may be replaced by any other known element having an equivalent function.

What is claimed is:

1. A method comprising binding biotin-binding proteins to a substrate by one or more patternings, adding one or more flow fields, adding F-actin so that the F-acitin is simultaneously bound and aligned to the biotin-binding proteins due to the one or more flow fields, adding myosin coated particles and a chemical fuel to transport cargo by the myosin coated particles in nanodevices and systems.

2. The method of claim 1 further comprising binding one or more biotyinylated caps to a selective end of the F-actin before the F-actin is bound to the biotin binding proteins.

3. The method of claim 1 wherein biotin binding protein is streptavidin, avidin, and neutravidin.

4. The method of claim 1 wherein substrate is glass, quartz, and plastic.

5. The method of claim 1 wherein patterning is accomplished by UV sensitivity photoresist and the use of a photo-biotin exposed to UV to activate the biotin with biotin binding proteins.

6. The method of claim 1 wherein patterning is selective patterning accomplished by special light sensitive polymers, soft and conventional lithography techniques, and scanning probe lithography techniques.

7. The method of claim 1 wherein myosin coated particle is bead, nanowire, and nanotube.

8. The method of claim 1 further comprising molding PDMS on one or more electrode substrates to create one or more microchannels before the binding of the biotin-binding proteins to a substrate for selective area transport.

9. The method of claim 1 further comprising the use of UV light exposure as a switch for the transport.

10. The method of claim 1 wherein the chemical fuel is ATP
